(19) United States
(12) Patent Application Publication
(10) Pub. No.: US 2003/0023079 A1
(43) Pub. Date: Jan. 30, 2003

(54) Polysaccharides derived from K5 polysaccharide having high anticoagulant and antithrombotic activities and process for their preparation

(76) Inventors: Pasqua Oreste, Milano (IT); Giorgio Zoppetti, Milano (IT)

Correspondence Address: MILLEN, WHITE, ZELANO & BRANIGAN, P.C., Arlington Courthouse Plaza I, Suite 1400, 2200 Clarendon Boulevard, Arlington, VA, 22201, US

(21) Appl. No.: 09/738,879

(22) Filed: Dec. 18, 2000

(30) Foreign Application Priority Data
Mar. 30, 2000 (IT) MI2000A000665

(51) Int. Cl. C08B 37/00; C12P 19/26

(52) U.S. Cl. 536/54; 435/84

(57) ABSTRACT

Glycosaminoglycans derived from K5 polysaccharide having high anticoagulant and antithrombotic activities are obtained by a process comprising the preparation of the K5 polysaccharide from Escherichia Coli, N-deacetylation/N-sulfation, C5 epimerisation, oversulfation, selective O-desulfation, selective 6-O sulfation and N-sulfation, in which said epimerisation is performed with the use of the enzyme glucuronosyl C5 epimerase in solution or immobilised in presence of specific divalent cations. New, particularly interesting compounds are obtained by controlling the reaction time in the O-desulfation step.

### BACKGROUND OF THE INVENTION

Glycosaminoglycans are biopolymers industrially extracted from different animal organs such as intestinal mucosa, lung and so on.

According to their structure glycosaminoglycans are divided into heparin, heparan sulfate, dermatan sulfate, chondroitin sulfate and hyaluronic acid. In particular heparin and heparan sulfate are composed by repeating disaccharide units formed by an uronic acid (L-iduronic acid or D-glucuronic acid) and by an amino sugar (glucosamine)

The uronic acid could be sulfated in position 2 and the glucosamine could be N-acetylated for the major part (heparan sulfate) or N-sulfated (heparin) and 6-O sulfated. Moreover glucosamine can contain one sulfate group in position 3.

Heparin and heparan sulfate are polydisperse molecules with a molecular weight ranging from 3,000 to 30,000 D.

Besides the known anticoagulant and antithrombotic activities, heparin exerts also antilipemic, antiproliferative, antiviral, anticancer and antiangiogenetic activity. To satisfy the major request of starting material for these new therapeutic areas a new alternative route of production different from the extractive ones from animal tissues is necessary. The natural biosynthesis of heparin in mammalians and its properties have been described by Lindahl et al. 1986 in Lane D. and Lindahl U. (Eds.) “Heparin-Chemical and Biological Properties; Clinical Applications”, Edward Arnold, London, pp.159-190 and Lindahl U., Feingold, D. S. and Rodén U. (1986) TIBS, 11, 221-225.

The sequence formed by the pentasaccharide region of linkage for Antithrombin III (ATIII) named active pentasaccharide that is the structure needed for the high affinity binding of heparin to ATIII, is fundamental for heparin activity. This sequence contains a unique glucosamine unit sulfated in position 3, that is not present in the other parts of the heparin chain. Beside the activity through ATIII, heparin exerts its anticoagulant and antithrombotic activity through the activation of heparin cofactor II (HCII) with a subsequent selective inhibition of thrombin. It is known that the minimum saccharidic sequence necessary for HCII activation is a chain containing at least 24 monosaccharides. (Tollefsen D. M. Seminars in Thrombosis and Haemostasis 16, 66-70 (1990)).

From the literature it is known that the capsular polysaccharide K5 isolated from the strain of Escherichia Coli described by Vann W. F., Schmidt M. A., Jann B., Jann K. (1981) in European Journal of Biochemistry 116, 359-364 shows the same sequence of heparin and heparan sulfate precursor (N-acetylheparosan). This compound was chemically modified as described by Lormeau et al. in the U.S. Pat. No. 5,550,116 and by Casu et al. (Carbohydrate Research 263, (1994), 271-284) or chemically and enzymatically modified as described by Jann et al. (WO 92/17509) and by Casu et al Carbohydrate Letters 1, 107-114 (1994). These modifications result in products with in vitro biological activities in coagulation of the same level of heparin of extracted from animal organs.

### SUMMARY OF THE INVENTION

We have found new glycosaminoglycans derived from K5 polysaccharide from Escherichia Coli with a molecular weight from 3,000 to 30,000, containing from 25% to 50% by weight of the chains with high affinity for ATIII and with a high anticoagulant and antithrombotic activity which is comprised between 1.5 and 4 if the results are expressed as ratio HCII/Anti-Xa activities with a prevalence of the activities which implies thrombin inhibition.

Said glycosaminoglycans are synthesised through a process comprising some steps of chemical and enzymatic modification and characterised by a step of epimerisation from D-glucuronic acid to L-Iduronic acid using the enzyme glucuronosyl C5 epimerase in solution or in immobilised form in presence of specific divalent cations, said enzyme being chosen from the group including recombinant glucuronosyl C5 epimerase, glucuronosyl C5 epimerase from murine mastocytoma and glucuronosyl C5 epimerase extracted from bovine liver and said divalent cations being chosen from the group comprising Ba, Ca, Mg and Mn.

More particularly, the process for the preparation of said glycosaminoglycans substantially comprises the following steps: (i) N-deacetylation/N-sulfation of the polysaccharide K5, (ii) partial C-5 epimerisation of the carboxyl group of the glucuronic acid moiety to the corresponding iduronic acid moiety, (iii) oversulfation, (iv) selective O-desulfation, (v) optional selective 6-O-sulfation, and (vi) N-sulfation. We have also found that different compounds are obtained by modulating the reaction time of the selective O-desulfation.

Moreover, we have found that, by carrying out the O-desulfation of the product obtained at the end of step (iii), whenever prepared according to the steps (i)-(iii) for a period of time of from 135 to 165 minutes, new compounds are obtained which show the best antithrombotic activity and a bleeding potential lower than that any other heparin-like glycosaminoglycan.

Finally, we have found that the best antithrombotic activity and the lowest bleeding potential depends on the O-sulfate group in the 3-position of the uronic moiety, especially of the iduronic one, and that such best ratio activity/bleeding is obtained when the 3-hydroxy group of the uronic, specially iduronic, moiety contains about 10% of sulfate groups.

### BRIEF DESCRIPTION OF THE DRAWINGS

FIG. 1 shows the ¹H-NMR spectrum of the K5 polysaccharide working standard.

FIG. 2 shows the ¹H-NMR spectrum of the K5 polysaccharide obtained in example 1a) and example 12.

FIG. 3 shows the ¹H-NMR spectrum of the purified K5 polysaccharide obtained in example 1 a) and example 12.

FIG. 4 shows the ¹³C-NMR spectrum of the N-sulphate K5 polysaccharide obtained in example 1 b) and example 12 i).

FIG. 5 shows the ¹H-NMR spectrum of the efficiency of the immobilised C-5 epimerase in example 1 c-1) and example 12 ii-1).

FIG. 6 shows the ¹H-NMR spectrum of the epimerised product obtained in example 1 c-2).

FIG. 7 shows the ¹³C-NMR spectrum of the oversulfate compound obtained in example 1 d).

FIG. 8 shows the ¹³C-NMR spectrum of the desulfated compound obtained in example 1 e).

FIG. 9 shows the ¹³C-NMR spectrum of the compound obtained in example 1 g).

FIG. 10 shows the chromatographic profile of the compound obtained in example 3.

FIG. 11A shows the chromatographic profile of the compound at high molecular weight obtained in example 10.

FIG. 11B shows the chromatographic profile of the compound at low molecular weight obtained in example 10.

FIG. 12 shows the ¹H-NMR spectrum of the epimerised product obtained in example 12 ii).

FIG. 13 shows the ¹³C-NMR spectrum of the oversulfated compound obtained in example 12 iii).

FIG. 14 shows the ¹³C-NMR spectrum of the desulfated compound obtained in example 12 iv).

FIG. 15 shows the ¹³C-NMR spectrum of the compound obtained in example 12 vi).

FIG. 16 shows the ¹³C-NMR spectrum of the desulfated compound obtained in example 13.

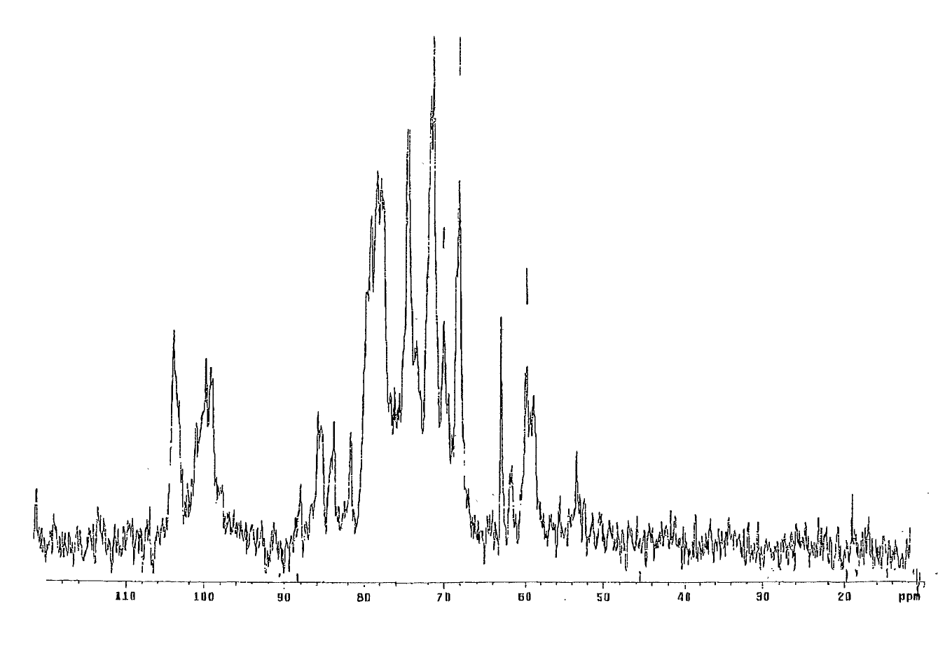

### DETAILED DESCRIPTION OF THE INVENTION

The present invention relates to glycosaminoglycans derived from K5 polysaccharide from Escherichia Coli (further simply named K5), obtained by a process which includes the following steps:

a) Preparation of K5 polysaccharide from Escherichia Coli

b) N-deacetylation/N-sulfation

c) C5 epimerisation

d) Oversulfation

e) Selective O-desulfation

f) Selective 6-O-sulfation (optional)

g) N-sulfation

The different steps of the process are detailed as follows.

a) Preparation of K5 Polysaccharide from Escherichia Coli

First a fermentation in flask is performed according to the patent MI99A001465 and using the following medium:

| | | |
|---|---|---|
| Defatted soy | 2 | gr/l |
| K₂HPO₄ | 9.7 | gr/l |
| KH₂PO₄ | 2 | gr/l |
| MgCl₂ | 0.11 | gr/l |
| Sodium citrate | 1 | gr/l |
| Ammonium solfate | 1 | gr/l |
| Glucose | 2 | gr/l |
| Water | 1,000 | ml |
| pH 7 3 | | |

The medium is sterilized at 120° C. for 20 minutes.

Glucose is prepared separately as a solution that is sterilised at 120° C. for 30) minutes and sterile added to the medium.

The flask is inoculated with a suspension of E. Coli cells Bi 8337/41 (O10.:K5:H4) from a slant containing tryptic soy agar and incubated at 37° C. for 24 hours under controlled stirring (160 rpm, 6 cm of run). The bacterial growth is measured counting the cells with a microscope.

In a further step, a Chemap-Braun fermentor with a volume of 14 liters containing the same medium above is inoculated with the 0.1% of the above flask culture and the fermentation is performed with 1 vvm aeration (vvm=air volume for liquid volume for minute) 400 rpm stirring and temperature of 37° C. for 18 hours. During the fermentation pH, oxygen, residual glucose, produced K5 polysaccharide and bacterial growth are measured.

At the end of the fermentation the temperature is raised to 80° C. for 10 minutes. The cells are separated from the medium by centrifugation at 10,000 rpm and the supernatant is ultrafiltrated through a SS316 (MST) module equipped with PES membranes with a nominal cut off of 800 and 10,000 D to reduce the volume to ⅕. Then K5 polysaccharide is precipitated adding 4 volumes of acetone at 4° C. and left to sediment for one night at 4° C. and finally is centrifuged at 10,000 rpm for 20 minutes or filtrated.

Then a deproteinisation using a protease of the type II from Aspergillus Orizae in 0.1M NaCl and 0.15 M EDTA at pH 8 containing 0.5% SDS (10 mg/l of filtrate) at 37° C. for 90 minutes is performed.

The solution is ultrafiltrated on a SS 316 module with a nominal cut off membrane of 10,000 D with 2 extractions with 1M NaCl and washed with water until the absorbance disappears in the ultrafiltrate. K5 polysaccharide is then precipitated with acetone and a yield of 850 mg/l of fermentor is obtained. The purity of the polysaccharide is measured by uronic acid determination (carbazole method), proton and carbon NMR, UV and protein content. The purity is above 80%.

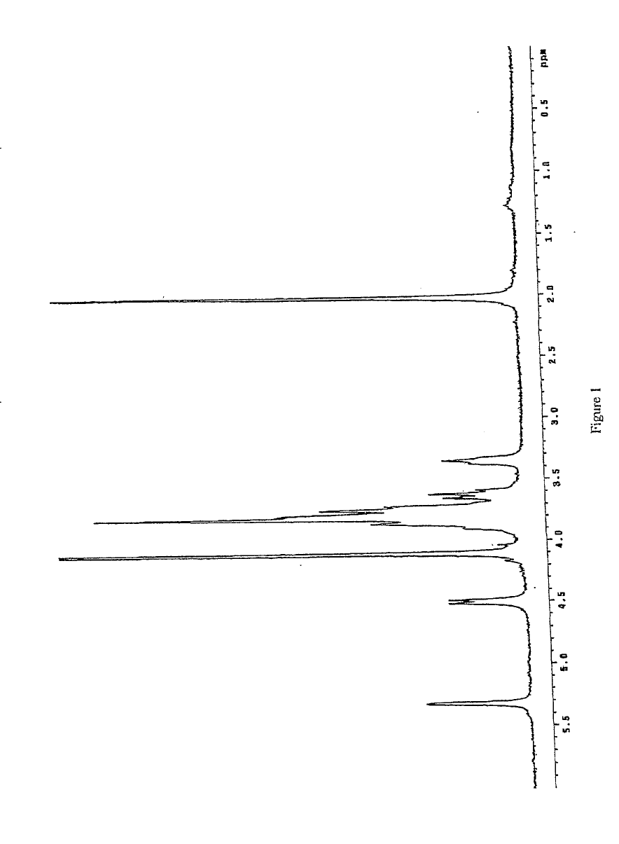

The so obtained polysaccharide is composed of two fractions with different molecular weight, 30,000 and 5,000 D respectively as obtained from the HPLC determination using a 75 HR Pharmacia column and one single fraction with retention time of about 9 minutes using two columns of Bio-sil SEC 250 in series (BioRad) and Na₂SO₄ as mobile phase at room temperature and flow rate of 0.5 ml/minute. The determination is performed against a curve obtained with heparin fractions with known molecular weight. The proton NMR is shown in FIG. 1.

To a 1% aqueous solution of the purified K5 polysaccharide Triton X-100 to a concentration of 5% is added. The solution is kept at 55° C. for 2 hours under stirring. The temperature is raised to 75° C. and during the cooling to room temperature two phases are formed.

On the upper phase (organic phase) the termic treatment with the formation of the two phases is repeated twice. The aqueous phase containing the polysaccharide is finally concentrated under reduced pressure and precipitated with ethanol or acetone The organic phase is discarded. The purity of the sample is controlled by proton NMR and results to be 95%.

The yield of this treatment is 90%.

### b) N-deacetylation/N-sulfation

10 gr of purified K5 polysaccharide are dissolved in 100-2,000 ml of 2N sodium hydroxide and left to react at 40-80° C. for the time necessary to achieve the complete N-deacetylation, which is never above 30 hours. The solution is cooled to room temperature and the pH brought to neutrality with 6N hydrochloric acid.

The solution containing the N-deacetylate K5 is kept at 20-65° C. and 10-40 gr of sodium carbonate are added together with 10-40 gr of a sulfating agent chosen among the available reagent such as the adduct pyridin sulfur trioxide, trimethyl amine sulfur trioxide and so on.

The addition of the sulfating agent if performed during a variable time till 12 hours. At the end of the reaction the solution is brought to room temperature, if necessary and to a pH of 7.5-8 with a 5% solution of hydrochloric acid.

The product is purified from salts with known technologies, for instance by diafiltration using a spiral membrane with 1,000 D cut off (prepscale cartridge-Millipore). The process is finished when the conductivity of the permeate is less than 1,000 μS, preferably less than 100 μS. The volume of the product obtained is concentrated till 10% polysaccharide concentration using the same filtration system as concentrator. If necessary the concentrated solution is dried with the known technologies.

The N/sulfate/N-acetyl ratio ranges from 10/0 to 7/3 measured by carbon 13 NMR.

### c) C5 Epimerisation

The step of C5 epimerisation according to the present invention can be performed with the enzyme glucuronosyl C5 epimerase (also called C5 epimerase) in solution or its immobilised form.

C5 epimerisation with the enzyme in solution.

From 1.2×10⁷ to 1.2×10¹¹ cpm (counts per minute) of natural or recombinant C5 epimerase, calculated according to the method described by Campbell P. et al Analytical Biochemistry 131, 146-152 (1983), are dissolved in 2-2,000 ml of 25 mM Hepes buffer at a pH comprised between 5.5 and 7.4, containing 0.001-10 gr of N-deacetylate N-sulfate K5 and one or more of the ions chosen among barium, calcium, magnesium, manganese at a concentration ranging from 10 and 60 mM. The reaction is performed at a temperature ranging from 30 and 40° C., preferably 37° C. for 1-24 hours. At the end of the reaction the enzyme is inactivated at 100° C. for 10 minutes. The product is purified by a passage on a DEAE resin or DEAE device Sartobind and unbound with 2M NaCl and finally desalted on a Sephadex G-10 resin or it is purified by precipitation with 2 volumes of ethanol and passage on a TR 120 H resin to make the sodium salt.

The product obtained shows an iduronic acid/glucuronic acid ratio between 40-60 and 60:40 calculated by ¹H-NMR as already described in the patent n. WO06/14425.

C5 Epimerisation with Immobilised Enzyme

The enzyme C5 epimerase, natural or recombinant, can be immobilised on different inert supports including resins, membranes or glass beads derivatised with reactive functional groups using the most common technologies of linkage for the enzymes such as cyanogen bromide, glutaraldehyde, carbodiimide or making the enzyme react with a ionic exchange resin or adsorbed on a membrane. According to the present invention the reactions of binding of the enzyme to the inert support are performed in presence of the substrate N-deacetylate N-sulfate K5 to avoid the active site of the enzyme to link with loss of activity.

The measure of the activity of the immobilised enzyme is performed recirculating the amount of N-deacetylate N-sulfate K5 that theoretically can be epimerised by that amount of cpm of immobilised enzyme onto a column of the immobilised enzyme in presence of 25 mM Hepes, 0.1M KCl, 0.01% Triton X-100 and 0.15 M EDTA pH 7.4 buffer at 37° C. overnight at a flow rate of 0.5 ml/minute. After the purification by DEAE chromatographic method and desalting on Sephadex G10 the product is freeze dried and the content of iduronic acid is calculated by proton NMR. The ratio iduronic acid/glucuronic acid shall be about 30/70.

20-1,000 ml of 25 mM Hepes buffer at a pH between 6 and 7.4 containing one or more ions chosen among barium, calcium, magnesium, manganese at a concentration between 10 and 60 mM and 0.001-10 gr N-deacetylate N-sulfate K5 kept at a temperature between 30 and 40° C., are recirculated at a flow rate of 30-160 ml/hour for 1-24 hours in a column containing from 1.2×10⁷ to 3×10¹¹ cpm equivalents of the enzyme immobilised on the inert support kept at a temperature from 30 to 40° C. At the end of the reaction the sample is purified with the same methods indicated in the epimerisation in solution.

The ratio iduronic acid/glucuronic acid of the product obtained ranges between 40:60 and 60.40.

d) Oversulfation

The solution containing the epimerised product of step c) at a concentration of 10% is cooled at 10° C. and passed through an IR 120 H⁺ column or equivalent (35-100 ml). Both the column and the container of the product are kept at 10° C. After the passage of the solution the resin is washed with deionised water until the pH of the flow through is more than 6 (about 3 volumes of deionised water). The acidic solution is kept to neutrality with a tertiary or quaternary amine such as tetrabuthylamonium hydroxide (15% aqueous solution) obtaining the ammonium salt of the polysaccharide. The solution is concentrated to the minimum volume and freeze dried. The product obtained is suspended in 20-500 ml of DMF or DMSO and added with 15-300 gr. of a sulfating agent such as the adduct pyridine —SO₃ in the solid form or in solution of DMF or DMSO. The solution is kept at 20-70° C., preferably between 40 and 60° C. for 2-24 hours.

At the end of the reaction the solution is cooled to room temperature and added with acetone saturated with sodium chloride till complete precipitation.

The precipitate is separated from the solvent by filtration, solubilised into the minimum amount of deionised water (for instance 100 ml) and added with sodium chloride to obtain a 0.2M solution. The solution is brought to pH 7.5-8 with 2N sodium hydroxide and added with acetone till complete precipitation. The precipitate is separated from the solvent by filtration. The solid obtained is dissolved into 100 ml of deionised water and purified from the residual salts by ultrafiltration as described in step b).

Part of the product is freeze dried for the structural analysis of the oversulfated product by ¹³C-NMR.

The content of sulfates per disaccharide of the product obtained is 2.0-3.5 calculated according to Casu B. et al. Carbohydrate Research 39 168-176 (1975). The position 6 of the glucosamine is sulfated at 80-95% and the position 2 is completely unsulfated. The other sulfate groups are present in position 3 of the amino sugar and 2 and 3 of the uronic acid.

e) Selective O-desulfation

The solution containing the product of the step d) is passed through a cationic exchange resin IR 120 H⁺ or equivalent (35-100 ml). After the passage of the solution the resin is washed with deionised water till the pH of the flow through is more than 6 (about 3 volumes of deionised water). The acidic solution is brought to neutrality with pyridine. The solution is concentrated to the minimum volume and freeze dried. The product obtained is treated with 20-2,000 ml of a solution of DMSO/methanol (9/1 V/V) and the solution is kept at 45-90° C. for 1-8 hours. Finally the solution is added with 10-200 ml of deionised water and treated with acetone saturated with sodium chloride to complete precipitation.

With the selective O-desulfation the sulfate groups in position 6 of the glucosamine are eliminated first, then the sulfates in position 3 and 2 of the uronic acid and finally the sulfate in position 3 of the amino sugar.

The solid obtained is purified by diafiltration as described in step b).

Some of the sample is freeze dried for the structural analysis by ¹³C-NMR.

If the content of the sulfate groups in position 6 of the amino sugar is more than 60%, calculated as described by Casu B. et al. Arzneimittel-forschung Drug Research 33-1 135-142 ( 1983) the step g) is performed. Otherwise the next step is performed.

f) Selective 6-O Sulfation (Optional)

The solution containing the product of step e) is treated as described in step d) to obtain the tertiary or quaternary ammonium salt, but performing the reaction at 20-25° C.

The ammonium salt is suspended in 20-500 ml of DMF. The suspension is cooled to 0° C. and treated with an amount of sulfating agent such as the adduct pyridine-SO₃ calculated in function of the percentage of the sulfate in position 6 of the amino sugar to be inserted taking in account a minimum of 60% of 6-O sulfation calculated as described above. The quantity of sulphating agent is comprised between two and ten equivalents of the hydroxyl groups to be sulfated. The sulfating agent is added one step or with several additions in a total time of 20 minutes.

The sulfating agent can be in powder or dissolved in a small amount of DMF

The solution is kept at 0-5° C. for 0.5-3 hours. The solution is then added with acetone saturated with sodium chloride in the right amount to complete the precipitation. The solid obtained is purified by diafiltration as described in step b).

A small amount is freeze dried for the structural analysis by ¹³C-NMR.

If the content of 6-O sulfate groups calculated by NMR is less than 60%, step f) is repeated.

g) N-sulfation

The solution obtained in step f) or, eventually, in step e) is treated as described in step b) for the N-sulfation.

The product of the present invention obtained from step d) to step g) can be chemically depolymerised as described in patent WO8203627, preferably after step g).

The glycosaminoglycans obtained with the process of the invention are characterised by proton and carbon 13 NMR and by biological tests like anti-Xa, aPTT, HCII, Anti-IIa and affinity for ATIII.

The product obtained can be fractionated by chromatography on resin or ultrafiltration obtaining low molecular weight fractions from 2,000 to 8,000 D and high molecular fractions from 25,000 to 30,000 D or it can be depolymerised with controlled known technologies such as nitrous acid deamination as described in the patent n. WO08203627.

The typical characteristics of molecular weight and biological activity of the glycosaminoglycans obtained with the process of the invention (IN-2018 UF and IN-2018 LMW) are indicated in table 1 in comparison with unfractionated heparin (4^{th} International Standard) and LMW heparin (1^{st} International Standard).

The molecular weight is calculated as indicated in references. The molecular weights can be different from those of the starting polysaccharide due to the reaction conditions of the process of the invention.

The activities indicated in rows 4, 5 and 6 are relative values in comparison with heparin taken as 100. The data of column 5 and 6 represent the range of values for the products prepared according to the process of the present invention.

TABLE 1
Biological activity of the product obtained by the described process:

| | Unfractionated | | | | |
|---|---|---|---|---|---|
| | heparin | LMW heparin | | IN-2018 | |
| Sample | (4^{th} int. Standard) | (1^{st} int. Standard) | IN-2018 UF | LMW | |
| 1 | Anti Xa | 100 | 54 | 70-250 | 40-100 |
| 2 | APTT | 100 | 30 | 40-90 | 25-80 |
| 3 | HCII | 100 | nd. | 300-500 | 100-200 |
| 4 | Anti IIa | 100 | 33 | 100-600 | 20-210 |
| 5 | Mean | 13,500 | 4,500 | 18,000-30,000 a) | 4,000- |
| | molecular | | | 10,000-20,000 b) | 8,000 |
| | weight | | | | |
| 6 | Affinity for ATIII | 32% | n.d. | 25-50 | 20-40 |

(n.d. = not determined)

References

1. Thomas D. P. et al. Thrombosis and Haemostasis 45 214 (1981) against the 4^{th} International Standard of Heparin

2. Andersson et al. Thrombosis Research 9 575 (1976) against the 4^{th} International Standard of Heparin

3. The test is performed mixing 20 μl of a solution of 0.05 PEU/ml of HCII (Stago) dissolved in water with 80 μl of a solution of the sample under examination at different concentrations and 50 μl of Thrombin (0.18 U/ml-Boheringer) in 0.02M tris buffer pH 7.4 containing 0.15 M NaCl and 0.1% PEG-6,000. The solution is incubated for 60 seconds at 37° C., then 50 μl of 1 mM chromogenic substrate Spectrozyme (American Diagnostic) are added.

The reaction is continuously recorded for 180 seconds with determinations every second at 405 nm using an automatic coagulometer ACL 7000 (Instrumentation Laboratory).

4. Test is performed mixing 30 μl of a solution containing 0.5 U/ml of ATIII (Chromogenix) dissolved in 0.1M tris buffer pH 7.4 with 30 μl of a solution of the sample under examination at different concentrations and 60 μl of thrombin (5.3 nKat/ml-Chromogenix) in 0.1 M tris buffer pH 7.4. The solution is incubated for 70 seconds at 37° C., then 60 μl of 0.5 mM chromogenic substrate S-2238 (Chromogenix) in water are added. The reaction is continuously recorded for 90 seconds with determinations every second at 405 run using an automatic coagulometer ACL 7000 (Instrumentation Laboratory).

5. Harenberg and De Vries J. Chromatography 261 287-292 (1983)

a) using a single column (Pharmacia 75BR)

b)using two columns (BioRad Bio-sil SEC250)

6. H\ddot{oo}k M. et al. Febs Letters 66 90-93 (1976)

From the table it is evident that the product obtained with the present process shows comparable activity with the extractive heparin in the anti-Xa test (1) and reduced global anticoagulant activity (2) while the values of the tests which implies inhibition of thrombin are markedly higher (3, 4). These characteristics are predictive of higher antithrombotic properties and less side effects such as the bleeding effect of the product obtained compared to the extractive heparin.

Due to their characteristics the glycosaminoglycans of the present invention can be used alone of in combination with acceptable pharmaceutical eccipients or diluents, for the anticoagulant and antithrombotic treatment.

In consequence the present invention comprises also the compositions containing a suitable amount of said glycosaminoglycans in combination with pharmaceutically acceptable eccipients or diluents.

Finally the present invention refers to the effective amount of said glycosaminoglycans for the anticoagulant and antithrombotic treatment.

According to an advantageous method, in a process for the preparation of K5 glycosaminoglycans comprising the steps (i)-(vi) above it is possible to modulate the activity of the obtained final compound by controlling the reaction time of step (iv), at a given temperature.

Thus, more particularly, the present invention provides a process for the preparation of K5 glycosaminoglycans comprising the steps of (i) N-deacetylation/N-sulfation of the polysaccharide K5, (ii) partial C-5 epimerisation of the carboxyl group of the glucuronic acid moiety to the corresponding iduronic acid moiety, (iii) oversulfation, (iv) selective O-desulfation, (v) optional selective 6-O-sulfation, and (vi) N-sulfation, in which step (iv) comprises treating the oversulfated product obtained at the end of step (iii) with a mixture methanol/dimethyl sulfoxide for a period of time of from 135 to 165 minutes.

Preferably, said period of time is of about 150 minutes.

The product of the present invention obtained from step iii) to step vi) can be chemically depolymerised as described in patent WO8203627, preferably after step vi).

According to a preferred embodiment, the treatment of the supersulfated product obtained at the end of step (iii) with a mixture methanol/dimethyl sulfoxide is made for a period of time of about 150 minutes at a temperature of about 60° C.

According to this advantageous method, from the oversulfated products, whenever prepared according to the steps (i)-(iii), new glycosaminoglycans are obtained which show the best antithrombotic activity and a bleeding potential lower than that of any other heparin-like glycosaminoglycan. These glycosaminoglycans, have the structure I as illustrated hereinbelow.

Thus, more particularly, the present invention concerns novel glycosaminoglycans constituted by a mixture of chains of the general structure:

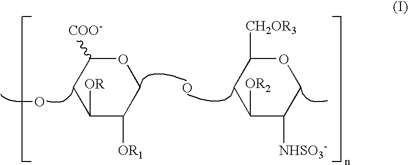

wherein at least 40% of the uronic moieties are those of iduronic acid, R, R₁, R₂ and R₃ represent a hydrogen atom or a SO₃⁻ group and n is an integer of from 3 to 100, from about 67% to about 60% of R, R₁, R₂ and R₃ being hydrogen and the remaining being SO₃⁻ groups distributed as follows: from about 85 to about 100% in R₃, from about 25 to about 30% in R₂, from about 25 to about 40% in R₁ and from about 10% in R, the sulfation degree being from about 2.30 to about 2.6, and the corresponding cation being a pharmaceutically acceptable one.

Advantageous low molecular weight glycosaminoglycans are constituted by a mixture of chains in which at least 80% of said chains have the structure I wherein n is from 3 to 15.

Among these low molecular weight glycosaminoglycans, those in which said mixture of chains has a molecular weight distribution ranging from about 2,000 to about 10,000, with a mean molecular weight of from about 4,000 to about 8,000 are preferred.

A particularly preferred, low molecular weight glycosaminoglycan having the above molecular weight distribution is a mixture of chains having the structure I in which at least 50% of the uronic moieties being those of iduronic acid and

R is for about 80% hydrogen and for about 20% a SO₃⁻ group;

R₁ is for about 60% hydrogen and for about 40% a SO₃⁻ group;

R₂ is for about 70% hydrogen and for about 30% a SO₃⁻ group:

R₃ is for about 10% hydrogen and for about 90% a SO₃⁻ group;

the sulfation degree being about 2.5.

Advantageous high molecular weight glycosaminoglycans are constituted by a mixture of chains in which at least 80% of said chains have the structure I wherein n is from 20 to 100.

Among these glycosaminoglycans, those in which said mixture of chains has a molecular weight distribution ranging from about 9,000 to about 60,000, with a mean molecular weight of from about 12,000 to about 30,000 are preferred.

A particularly preferred, high molecular weight glycosaminoglycan having the above molecular weight distribution is a mixture of chains having the structure I in which at least 50% of the uronic moieties being those of iduronic acid and

R is for about 90% hydrogen and for about 10% a SO₃⁻ group;

R₁ is for about 60% hydrogen and for about 40% a SO₃⁻ group;

R₂ is for about 70% hydrogen and for about 30% a SO₃⁻ group;

R₃ is for about 5% hydrogen and for about 95% a SO₃⁻ group;

the sulfation degree being about 2.5.

Advantageous pharmaceutically acceptable cations are those derived from alkaline metals alkaline-earth metals, ammonium, (C₁C₄)trialkylammonium, aluminium and zinc, sodium and calcium ions being particularly preferred.

The percent of the sulfate group in the 3-position of the uronic moiety has been determined by 13C-NMR on the compound obtained after step e).

Particularly the present invention provides pharmaceutical compositions for an anticoagulant or antithrombotic treatment comprising a glycosaminoglycan constituted by a mixture of chains having the structure I, as illustrated above, as an active ingredient, and a pharmaceutical carrier. In said pharmaceutical compositions, for intravenous, subcutaneous or topical use, the active ingredient is present in an effective dose for the treatment of diseases caused by disorders of the coagulation system, such as arterial or venous thrombosis and haematomas or as anticoagulant agents useful to prevent coagulation during surgical operations.

In preparations for intravenous or subcutaneous use, the glycosaminoglycans having the structure I, as illustrated above, are dissolved in water, if necessary in the presence of a buffer and the solution is introduced in vials or syringes under sterile conditions. Unit doses of said pharmaceutical compositions contain from 5 to 100 mg advantageously from 20 to 50 mg of active ingredient dissolved in 0.1 to 2 ml of water.

In compositions for topical use, the glycosaminoglycans having the structure I, as illustrated above, are mixed with pharmaceutical acceptable carriers or diluents known in the art for the preparation of gels, creams, ointments, lotions or solutions to be sprayed. In said compositions, the active ingredient having the structure I, as illustrated above, is present in a concentration of from 0.01% to 15% by weight advantageously.

Advantageous pharmaceutical compositions comprise, as an active ingredient, a glycosaminoglycan constituted by a mixture of chains of the general structure I, as illustrated above, in which the counter-ion is a pharmaceutically acceptable one advantageously a cation selected from the group consisting of alkaline metal alkaline-earth metal, ammonium, (C₁-C₄)trialkylammonium, aluminium and zinc ions and preferably the sodium or calcium ion, and a pharmaceutical carrier. Among these advantageous glycosaminoglycans, those which contain at least 80% of chains of formula I wherein n is from 3 to 15 or from 20 to 100 are preferred active ingredients, those in which the mixture of chains has a molecular weight distribution ranging from about 2,000 to about 10,000, with a mean molecular weight of from about 4,000 to about 8,000 or a molecular weight distribution ranging from about 9,000 to about 60,000, with a mean molecular weight of from about 12,000 to about 30,000, being particularly preferred.

To illustrate the invention the following examples are reported:

### EXAMPLE 1

The example 1 is performed according to the following steps:

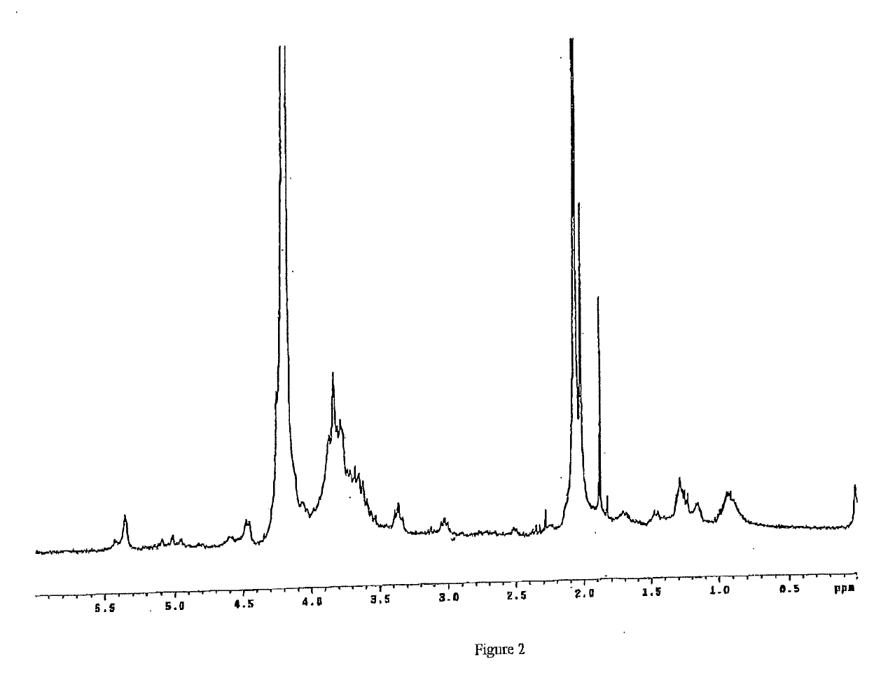

a) 10 gr. of polysaccharide obtained by fermentation as described in the patent MI99A001465 with a purity of 80% (FIG. 2) are dissolved in deionised water to obtain a 1% solution. Triton X-100 is added to reach a concentration of 5% and the solution is kept at 55° C. for 2 hours under stirring.

The solution is brought to 75° C. and kept at this temperature till a homogeneous turbid system is obtained and then the solution is rapidly cooled to room temperature. During the cooling two phases are formed.

Said thermal treatment is repeated twice on the upper phase (organic phase). The aqueous phase containing K5 polysaccharide is finally 1/10 concentrated under reduced pressure and precipitated with acetone or ethanol. The organic phase is discarded.

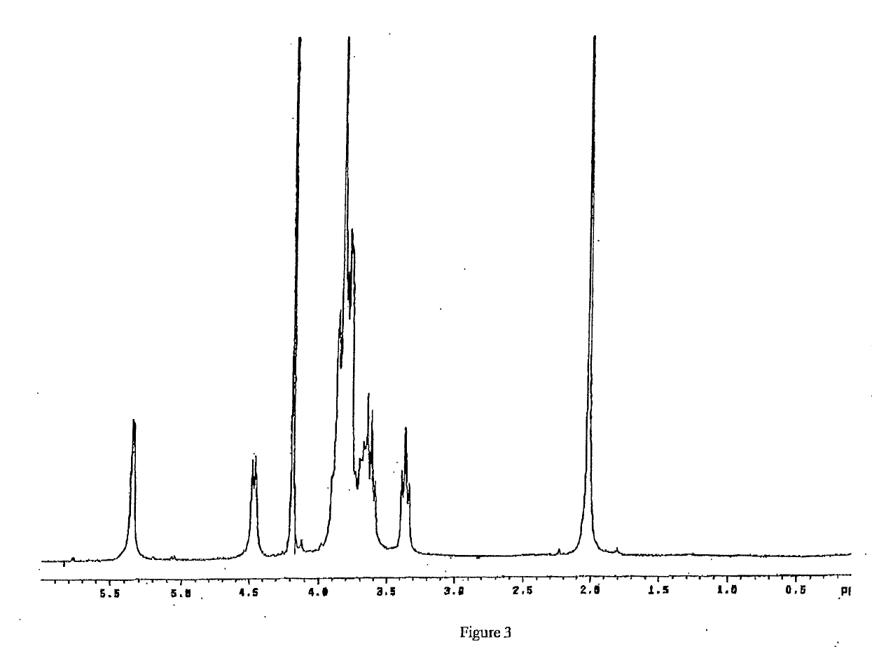

The product obtained is K5 polysaccharide with 90% purity detected by proton NMR (FIG. 3) compared to the spectrum of the working standard (FIG. 1).

b) The product obtained in step a) is dissolved in 1,000 ml of 2 N sodium hydroxide and kept at 60° C. for 18 hours. The solution is cooled to room temperature and then brought to neutral pH with 6N hydrochloric acid. N-deacetylate K5 polysaccharide is obtained.

The solution containing the N-deacetylate K5 is kept at 40° C. and added with 10 gr sodium carbonate in one step and 10 gr. of adduct pyridine sulfur trioxide in 10 minutes. At the end of the reaction the solution is cooled to room temperature and then brought to pH 7.5-8 with a 5% hydrochloric acid solution.

The product obtained, N-deacetylated N-sulfate K5 polysaccharide, is purified from salts by diafiltration using a 1,000 D cut off spiral membrane (prepscale cartridge—Millipore). The purification process is stopped when the conductivity of the permeate is less than 100 μS.

The product retained by the membrane is concentrated to 10% polysaccharide using the same diafiltration system and then is freeze dried.

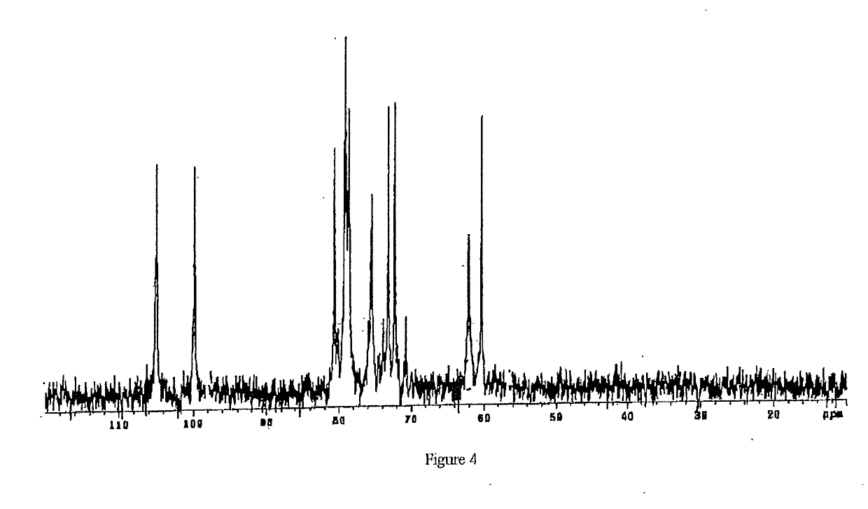

The ratio N-sulfate/N-acetyl in the product obtained is 9.5/05. measured by carbon 13 NMR (FIG. 4).

c) 1-Preparation of the Immobilised C5 Epimerase

To 5 mg of recombinant C5 epimerase obtained according to the patent WO98/48006 corresponding to 1.2×10 cpm (counts per minutes) dissolved in 200 ml of 25 mM Hepes buffer pH 7.4, containing 0.1 M KCl, 0.1% Triton X-100 and 15 mM EDTA, 100 mg of N-deacetylate N-sulfate K5 obtained as described in step b) are added. The solution is diafiltrated with a 30,000 D membrane at 4° C. till disappearance of N-deacetylate N-sulfate K5 in the permeate. To the solution rententated by the membrane the buffer is changed by diafiltration against 200 mM NaHCO₃ at pH 7 and, after concentration to 50 ml, 50 ml of CNBr activated Sepharose 4B resin are added and kept to react overnight at 4° C.

At the end of the reaction the amount of residual enzyme in the supernatant is measured with the Quantigold method (Diversified Biotec) after centrifugation. The enzyme in the supernatant is absent, showing that with the method described the enzyme is 100% immobilised.

To occupy the sites still available the resin is washed with 100 mM tris pH 8.

To measure the activity of the immobilised enzyme an amount of immobilised enzyme theoretically correspondent to 1.2×10⁷ cpm is loaded into a column. In the column obtained 1 mg of N-deacetylate N-sulfate K5 obtained as described in step b) dissolved in 25 mM Hepes, 0.1M KCl, 0.015 M EDTA, 0.01% Triton X-100, pH 7.4 buffer is dissolved, recirculating it through said column at 37° C. overnight at a flow rate of 0.5 ml/minute.

After purification by DEAE chromatographic system and desalting on a Sephadex G-10 the sample is freeze dried and analysed for its content in iduronic acid by proton NMR technique as already described in the patent n. WO96/14425.

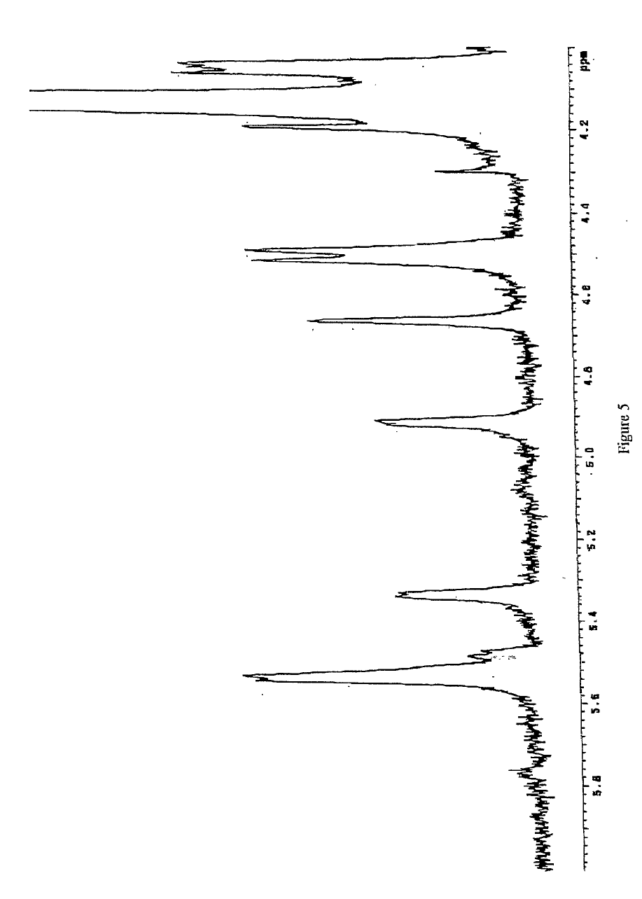

The ratio iduronic acid/glucuronic acid is 30/70. (FIG. 5)

2-Epimerisation

10 gr of the N-deacetylate N-sulfate K5 polysaccharide are dissolved in 600 ml of 25 mM Hepes buffer pH 6.5 containing 50 mM CaCl₂. The solution obtained is recirculated through a column of 50 ml containing the resin with the immobilised enzyme.

This reaction is performed at 37° C. with a flow rate of 200 ml/hour for 24 hours.

The product obtained is purified by ultrafiltration and precipitation with ethanol. The pellet is dissolved in water at 10% concentration.

An epimerised product is obtained with a ratio iduronic acid/glucuronic acid 48/52 against a ratio 0/100 of the starting material.

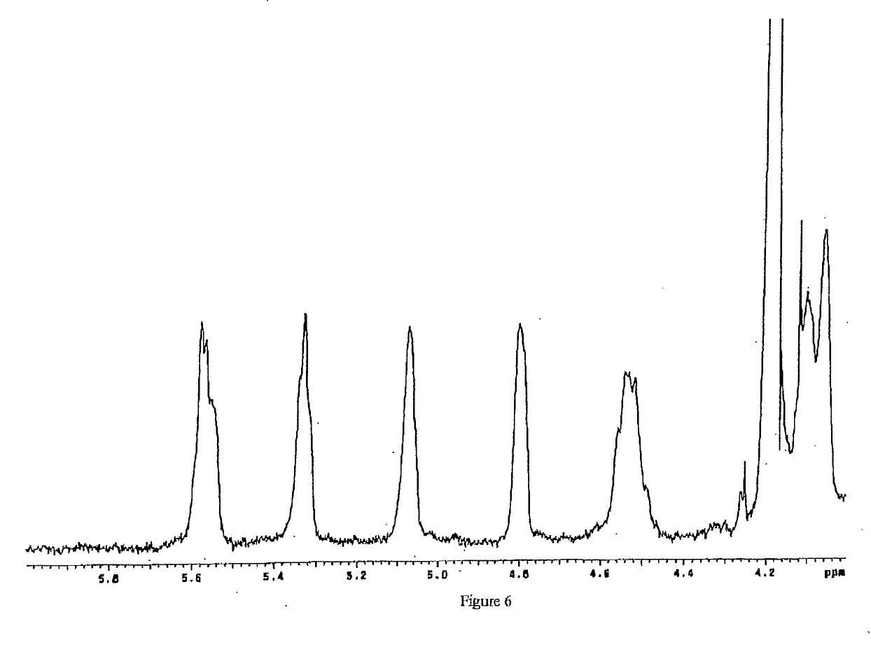

The percentage of epimerisation is calculated by ¹H-NMR (FIG. 6).

The yield calculated measuring the uronic acid content against standard by the carbazole method (Bitter and Muir Anal. Biochem. 39 88-92 (1971)) is 90%.

d) The solution containing the epimerised product with 10% concentration obtained in step c) is cooled to 10° C. with a cooling bath and then applied onto a IR 120 H⁺ cationic exchange resin (50 ml). Both the column and the container of the eluted solution are kept at 10° C. After the passage of the solution the resin is washed with 3 volumes of deionised water. The pH of the flow through is more than 6. The acidic solution is brought to neutrality with an aqueous solution of 15% tetrabutylammoniun hydroxide. The solution is concentrated to 1/10 of the volume in a rotating evaporator under vacuum and freeze dried.

The product is suspended in 200 ml of DMF and added with 150 gr of the adduct pyridine-SO₃ dissolved in 200 ml of DMF. The solution is kept at 45° C. for 18 hours.

At the end of the reaction the solution is cooled to room temperature and added with 1,200 ml of acetone saturated with sodium chloride.

The pellet obtained is separated from the solvent by filtration, dissolved with 100 ml of deionised water and sodium chloride is added to 0.2M concentration. The solution is brought to pH 7.5-8 with 2N sodium hydroxide and 300 ml of acetone are added. The pellet is separated by filtration. The solid obtained is solubilised with 100 ml deionised water and purified from the residual salts by diafiltration as described in step b).

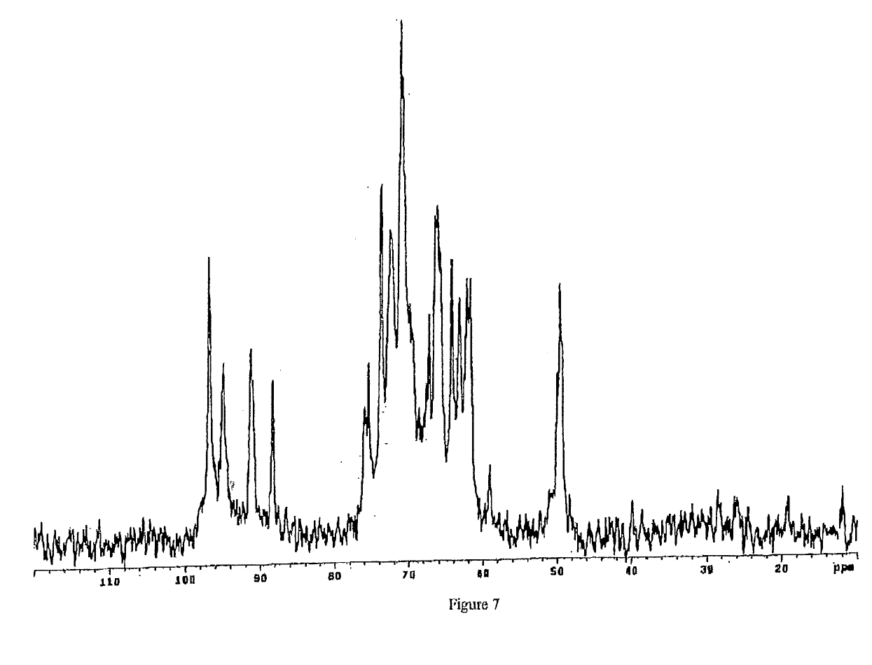

The ¹³C-NMR analysis on a dried small amount of the oversulfated product is shown in FIG. 7.

e) The solution containing the product of step d) is passed onto a IR 120 H⁺ cationic exchange resin (50 ml). After the passage of the solution the resin is washed with 3 volumes of deionised water. The pH of the flow through is more than 6. The acidic solution is brought to neutrality with pyridine. The solution is concentrated to 1/10 of the volume in a rotating evaporator at 40° C. under vacuum and freeze dried.

The product obtained as pyridine salt is added with 500 ml of a solution of DMSO/methanol (9/1 V/V). The solution is kept at 60° C. for 3.5 hours and then added with 50 ml deionised water and finally treated with 1,650 ml acetone saturated with sodium chloride.

The solid obtained is purified by diafiltration as described in step b) and a solution at 10% concentration is obtained.

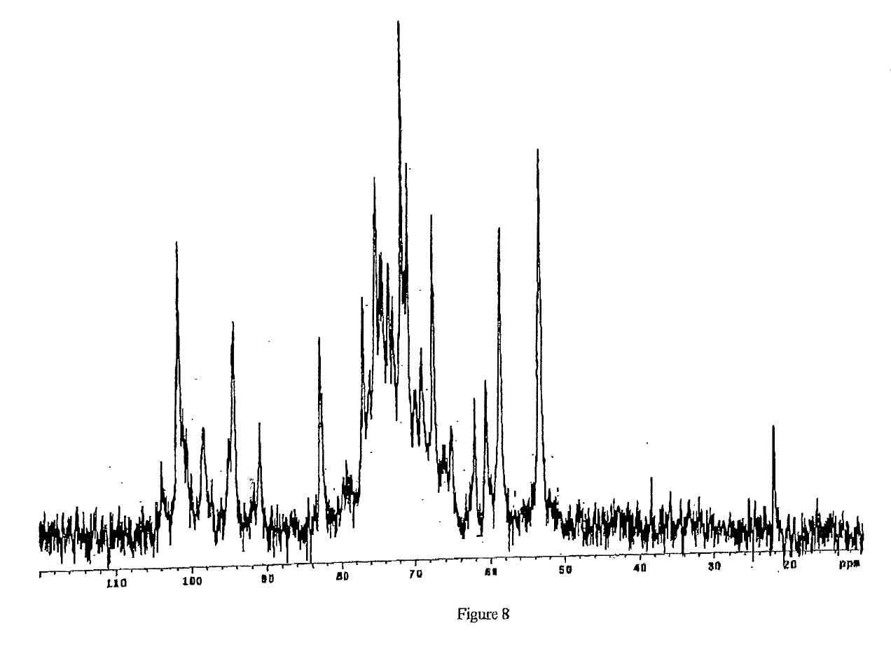

The ¹³C-NMR analysis on a dried small amount in FIG. 8 shows a content of sulfate groups in position 6 of the amino sugar of 35%.

f) The solution containing the product of step e) is passed onto a TR 120 H⁺ cationic exchange resin (50 ml). After the passage of the solution the resin is washed with 3 volumes of deionised water. The pH of the flow through is more than 6. The acidic solution is brought to neutrality with an aqueous solution of 15% tetrabutylammoniun hydroxide. The solution is concentrated to 1/10 of the volume in a rotating evaporator under vacuum and freeze dried. The product as tetrabutylammonium salt is suspended in 200 ml DMF. The suspension is cooled to 0° C. and treated with 40 gr. of the adduct pyridine-SO₃ dissolved in 100 ml DMF.

The sulfating agent is added one step.

The solution is kept at 0° C. for 1.5 hours and then is treated with 750 ml acetone saturated with sodium chloride.

The solid obtained is purified by diafiltration as described in step b).

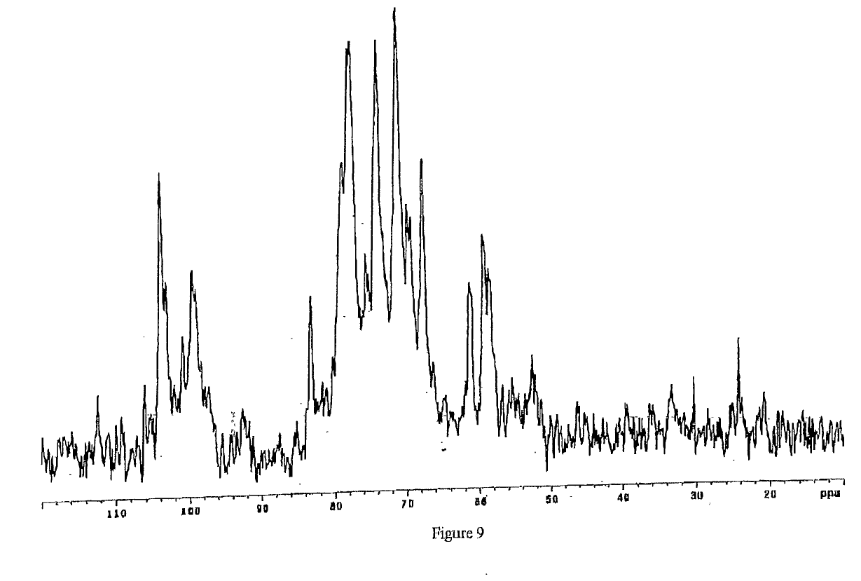

g) The solution of step f) is treated as described in step b) for N-sulfation. The ¹³C-NMR on a dried small amount of the product obtained is shown in FIG. 9.

The product obtained shows the physico-chemical and biological characteristics of table 2—line 3 compared with the 4^{th} International Standard Heparin and the 1^{st} International Standard Low Molecular Weight Heparin.

### EXAMPLE 2

Example 1 was repeated but in step c) the immobilised enzyme C5 epimerase extracted from murine mastocytoma was used as described by Jacobsson et al. J. Biol. Chem. 254 2975-2982 (1979), in a buffer containing 40 mM CaCl₂ pH 7.4.

The product obtained has a ratio iduronic acid/glucuronic acid of 59.5:40.5 and the characteristics described in table 2 line 4.

### EXAMPLE 3

Example 1 was repeated but in step c) the immobilised enzyme C5 epimerase extracted from bovine liver was used as described in WO96/14425 with a reaction buffer at pH 7.4 and reaction time of 32 hours. Moreover in step e) the reaction time was 4 hours.

The product obtained has a ratio iduronic acid/glucuronic acid of 55.4:44.6 and the characteristics described in table 2 line 5.

### EXAMPLE 4

Example 1 was repeated but in step c) the recombinant enzyme C5 epimerase in solution was used using for the epimerisation 10 gr N-deacetylate N-sulfate K5 dissolved in 1,000 ml of 25 mM Hepes buffer pH 6.5 containing 50 mM CaCl₂. To this solution 1.5×10¹¹ cpm equivalents of recombinant enzyme described in example 1 are added. The solution is kept at 37° C. for 24 hours. The solution is then treated at 100° C. for 10 minutes to denaturate the enzyme and finally is filtered on a 0.45μ filter to obtain a clear solution containing the product. The product obtained is then purified by diafiltration and precipitation with ethanol or acetone. The pellet is dissolved in water at 10% concentration and treated like in example 1 keeping the reaction time of step e) for 2 hours.

The product obtained has a ratio iduronic acid/glucuronic acid of 56:44 and the characteristics described in table 2 line 6.

### EXAMPLE 5

Example 4 is repeated using in step c) the enzyme from murine mastocytoma described in example 2, in solution, with the reaction buffer at pH 7.4 containing 40 mM BaCl₂ and performing the reaction for 18 hours. Moreover in step e) the reaction time is 3 hours.

The product obtained has a ratio iduronic acid/glucuronic acid of 40.1:59.9 and the characteristics described in table 2 line 7.

### EXAMPLE 6

Example 4 is repeated using in step c) the enzyme from bovine liver of example 3, in solution, with the reaction buffer containing 12.5 mM MnCl₂ and performing the reaction for 14 hours. Moreover in step e) the reaction time is 4 hours.

The product obtained has a ratio iduronic acid/glucuronic acid of 44.3:55.7 and the characteristics described in table 2 line 8.

### EXAMPLE 7

Example 4 is repeated using in step c) a reaction buffer at pH 7.4 containing 37.5 mM MgCl₂ and performing the reaction for 16 hours. Moreover in step e) the reaction time is 4 hours.

The product obtained has a ratio iduronic acid/glucuronic acid of 47.5:52.5 and the characteristics described in table 2 line 9.

### EXAMPLE 8

Example 3 is repeated using in step c) a reaction buffer at pH 7.0 containing 10 mM MgCl₂, 5 mM CaCl₂, 10 mM MnCl₂ and performing the reaction for 24 hours. Moreover in step e) the reaction time is 3 hours.

The product obtained has a ratio iduronic acid/glucuronic acid of 44.8:55.2 and the characteristics described in table 2 line 10.

### EXAMPLE 9

Example 6 is repeated using in step c) a reaction buffer at pH 7.4 containing 10 mM MgCl₂, 5 mM CaCl₂, 10 mM MnCl₂ and performing the reaction for 24 hours. Moreover in step e) the reaction time is 3 hours.

The product obtained has a ratio iduronic acid/glucuronic acid of 52:48 and the characteristics described in table 2 line 11.

### EXAMPLE 10

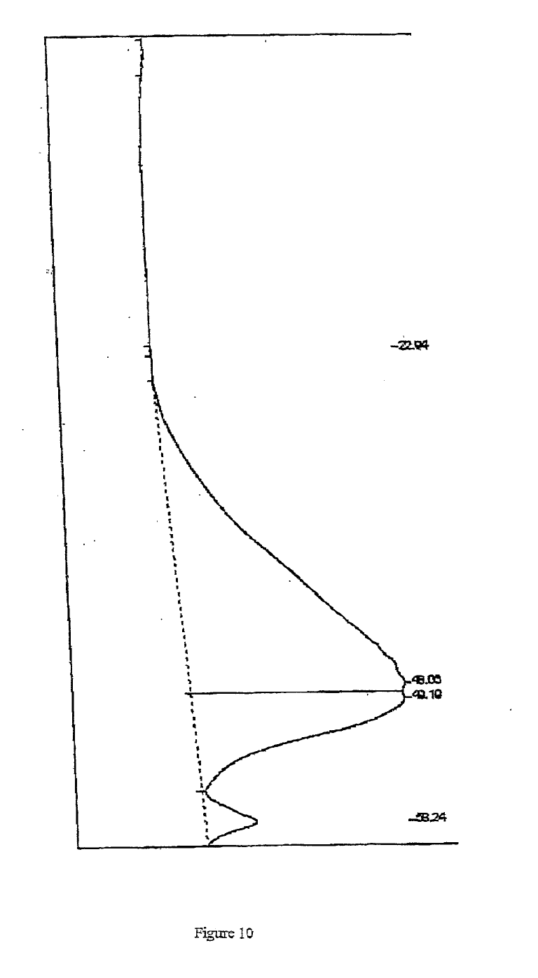

The sample obtained in example 3 having a molecular weight distribution calculated according to Harenberg and De Vries J. Chromatography 261 287-292 (1983) (FIG. 10) is fractionated by gel filtration. In particular 1 gr. of product is dissolved in 20 ml of 1M NaCl solution and loaded onto a column containing 1,000 ml of Sephacryl HR S-400 resin (Amersham-Pharmacia). The column is then eluted with 2,000 ml of 1M NaCl solution and collected in 50 ml fractions by fraction collector (Gilson). After the determination of product content on each fraction by carbazole reaction (Bitter and Muir Anal Biochem. 39 88-92 (1971)) the fractions containing the sample are combined in fraction A and fraction B respectively corresponding to the high molecular weight and low molecular weight fraction. These fractions are concentrated at 10% of the volume by evaporator under vacuum and are desalted on a column containing 500 ml of Sephadex G-10 resin (Amersham-Pharmacia).

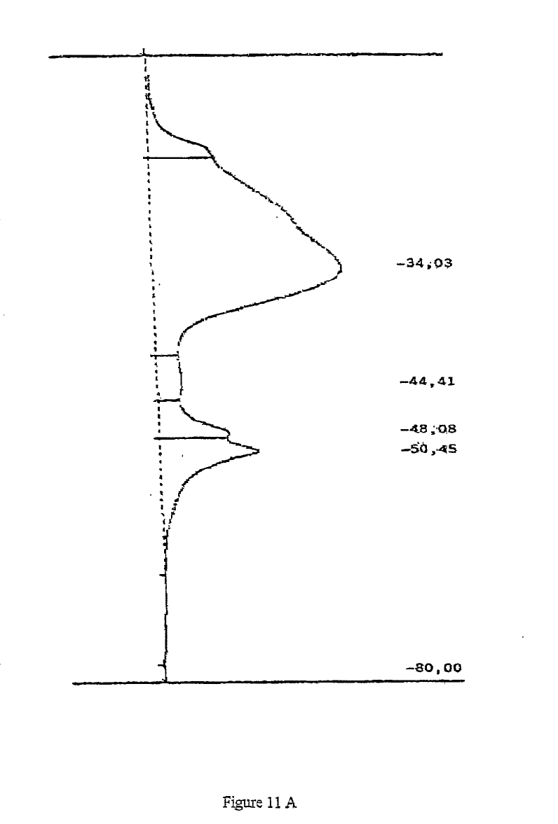

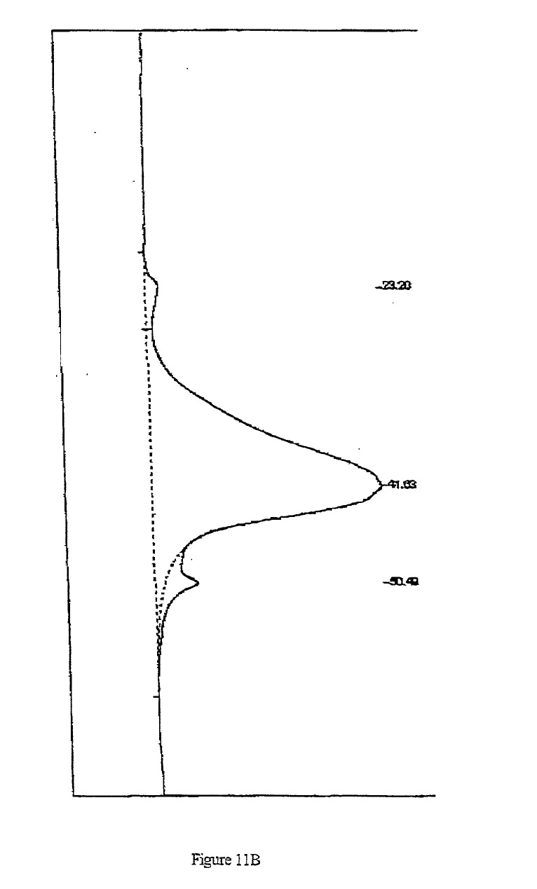

The solutions containing the desalted products are freeze dried obtaining fraction A and fraction B (FIG. 11A and FIG. 11B). The products obtained show the characteristics described in table 2 lines 12 and 13.

### EXAMPLE 11

The sample obtained in example 4 is degraded with nitrous acid in a controlled way as described in the patent WO 8203627. In particular 5 gr. of sample are dissolved in 250 ml of water and cooled to 4° C. with a thermostatic bath. The pH is brought to 2 with 1 N hydrochloric acid cooled at 4° C. and then 10 ml of a solution of 1% sodium nitrite are added. If necessary the pH is brought to 2 with 1N hydrochloric acid and is kept under slow stirring for 15 minutes. The solution is neutralised with 1N NaOH cooled at 4° C. Then 250 mg of sodium boro hydride dissolved in 13 ml of deionised water are added and the reaction is maintained for 4 hours. The pH is brought to 5 with 1N hydrochloric acid and the reaction kept for 10 minutes to destroy the excess of sodium boro hydride, and then neutralised with 1N NaOH. The product is recovered by precipitation with 3 volumes of ethanol and then dried in a vacuum oven. The product obtained shows the characteristics described in table 2 line 14.

TABLE 2
Anticoagulant and antithrombotic activity of the products obtained in the
described examples.

| | 1) Anti Xa | 2) APTT | 3) HCII | 4) Anti IIa | | 6) Affinity |
|---|---|---|---|---|---|---|
| | (%) | (%) | (%) | (%) | 5) MW | ATIII (%) |
| Unfractionated Hep (4^{th} | 100 | 100 | 100 | 100 | 13,500 | 32% |
| int STD | | | | | | |
| LMW heparin (1^{st} Int. | 84 | 30 | n.d. | 33 | 4,500 | n.d. |
| Std) | | | | | | |
| Example 1 | 76.6 | 43.4 | 256 | 118 | 15,200 | 29 |
| Example 2 | 94.3 | 57 | 294 | 208 | 13,500 | 29.5 |
| Example 3 | 112 | 88 | 346 | 223 | 14,600 | 28 |
| Example 4 | 157 | 71.5 | 362 | 600 | 22,500 a) | 29 |
| | | | | | 13,000 b) | |
| Example 5 | 150 | 70 | 352 | 213 | 24,000 a) | 31 |
| | | | | | 13,100 b) | |
| Example 6 | 150 | 79 | 335 | 333 | 23,000 a) | 33 |
| | | | | | 12,600 b) | |
| Example 7 | 120 | 92 | 346 | 247 | 13,000 a) | 29 |
| | | | | | 10,100 b) | |
| Example 8 | 153 | 75 | 332 | 240 | 22,500 a) | 34 |
| | | | | | 13,000 b) | |
| Example 9 | 157 | 71 | 346 | 233 | 23,000 a) | 35 |
| | | | | | 12,600 b) | |
| Example 10-A | 250 | 70.8 | 480 | 435 | 30,000 | 48 |
| Example 10-B | 43 | 77.7 | 145 | 27.3 | 7,600 | 24 |
| Example 11 | 97.5 | 55.5 | 230 | 210 | 5,400 | 25 |

(n.d. = not determined)

The references from 1) to 6) have the same meaning as for table 1.

From the table it is evident that the product obtained with the present process shows activities comparable to the extractive heparin in the Anti-Xa test (1) while the global anticoagulant activity is reduced (2) and the tests which refer to thrombin inhibition are markedly higher (3, 4). These characteristics of the product result in higher antithrombotic properties and lower side effects such as bleeding effect if compared to the extractive heparin.

### EXAMPLE 12

The example 12 is performed starting from 10 gr. of polysaccharide obtained by fermentation as described in the patent MI99A001465 with a purity of 80% (FIG. 2) are dissolved in deionised water to obtain a 1% solution. Triton X-100 is added to reach a concentration of 5% and the solution is kept at 55° C. for 2 hours under stirring. The solution is brought to 75° C. and kept at this temperature till a homogeneous turbid system is obtained and then the solution is rapidly cooled to room temperature. During the cooling two phases are formed.

Said termic treatment is repeated twice on the upper phase (organic phase). The aqueous phase containing K5 polysaccharide is finally 1/10 concentrated under reduced pressure and precipitated with acetone or ethanol. The organic phase is discarded.

The product obtained is K5 polysaccharide with 90% purity detected by proton NMR (FIG. 3) compared to the spectrum of the working standard (FIG. 1) and a retention time of 9 minutes on the HPLC analysis using two columns (Bio Rad Bio-sil SEC 250).

The process proceed according to the following steps:

i. The K5 polysaccharide is dissolved in 1,000 ml of 2 N sodium hydroxide and kept at 60° C. for 18 hours. The solution is cooled to room temperature and then brought to neutral pH with 6N hydrochloric acid. N-deacetylate K5 polysaccharide is obtained.

The solution containing the N-deacetylate K5 is kept at 40° C. and added with 10 gr sodium carbonate in one step and 20 gr. of adduct pyridine sulfur trioxide in 10 minutes. At the end of the reaction the solution is cooled to room temperature and then brought to pH 7.5-8 with a 5% hydrochloric acid solution.

The product obtained, N-deacetylated N-sulfate K5 polysaccharide, is purified from salts by diafiltration using a 1,000 D cut off spiral membrane (prepscale cartridge—Millipore). The purification process is stopped when the conductivity of the permeate is less than 100 μS.

The product retained by the membrane is concentrated to 10% polysaccharide using the same diafiltration system and then is freeze dried.

The ratio N-sulfate/N-acetyl in the product obtained is 9.5/0.5 measured by carbon 13 NMR (FIG. 4).

ii. 1-Preparation of the Immobilised C5 Epimerase

To 5 mg of recombinant C5 epimerase obtained according to the patent WO98/48006 corresponding to 1.2×10¹¹ cpm (counts per minutes) dissolved in 200 ml of 25 mM Hepes buffer pH 7.4. containing 0.1 M KCl, 0.1% Triton X-100 and 15 mM EDTA, 100 mg of N-deacetylate N-sulfate K5 obtained as described in step i) are added. The solution is diafiltrated with a 30,000 D membrane at 4° C. till disappearance of N-deacetylate N-sulfate K5 in the permeate. To the solution rententated by the membrane the buffer is changed by diafiltration against 200 mM NaHCO₃ at pH 7 and, after concentration to 50 ml, 50 ml of CNBr activated Sepharose 4B resin are added and kept to react overnight at 4° C.

At the end of the reaction the amount of residual enzyme in the supernatant is measured with the Quantigold method (Diversified Biotec) after centrifugation. The enzyme in the supernatant is absent, showing that with the method described the enzyme is 100% immobilised.

To occupy the sites still available the resin is washed with 100 mM tris pH 8. To measure the activity of the immobilised enzyme an amount of immobilised enzyme theoretically correspondent to 1.2×10⁷ cpm is loaded into a column. In the column obtained 1 mg of N-deacetylate N-sulfate K5 obtained as described in step b) dissolved in 25 mM Hepes, 0.1M KCl, 0.015 M EDTA, 0.01% Triton X-100, pH 7.4 buffer is dissolved, recirculating it through said column at 37° C. overnight at a flow rate of 0.5 ml/minute.

After purification by DEAE chromatographic system and desalting on a Sephadex G-10 the sample is freeze dried and analysed for its content in iduronic acid by proton NMR technique as already described in the patent application n. WO96/14425.

The ratio iduronic acid/glucuronic acid is 30/70. (FIG. 5)

2-Epimerisation

10 gr of the N-deacetylate N-sulfate K5 polysaccharide are dissolved in 600 ml of 25 mM Hepes buffer pH 7 containing 50 mM CaCl₂. The solution obtained is recirculated through a column of 50 ml containing the resin with the immobilised enzyme.

This reaction is performed at 30° C. with a flow rate of 200 ml/hour for 24 hours.

The product obtained is purified by ultrafiltration and precipitation with ethanol. The pellet is dissolved in water at 10% concentration.

An epimerised product is obtained with a ratio iduronic acid/glucuronic acid 54/46 against a ratio 0/100 of the starting material.

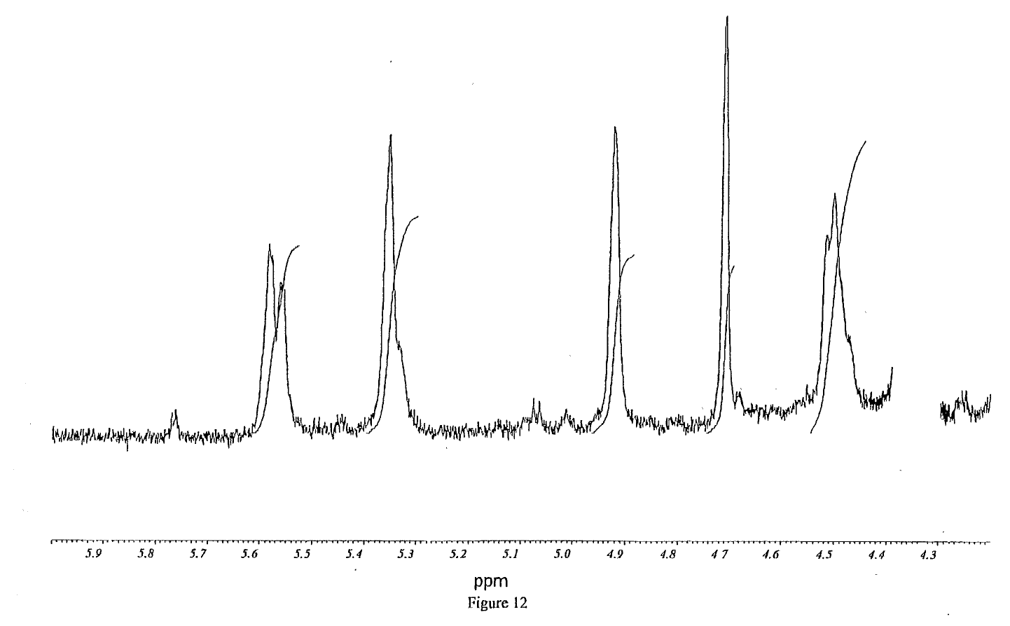

The percentage of epimerisation is calculated by ¹H-NMR (FIG. 12).

The yield calculated measuring the uronic acid content against standard by the carbazole method (Bitter and Muir Anal. Biochem. 39 88-92 (1971)) is 90%.

iii. The solution containing the epimerised product with 10% concentration obtained in step ii) is cooled to 10° C. with a cooling bath and then applied onto a IR 120 H⁺ cationic exchange resin (50 ml). Both the column and the container of the eluted solution are kept at 10° C. After the passage of the solution the resin is washed with 3 volumes of deionised water. The pH of the flow through is more than 6. The acidic solution is brought to neutrality with an aqueous solution of 15% tetrabutylammoniun hydroxide. The solution is concentrated to 1/10 of the volume in a rotating evaporator under vacuum and freeze dried.

The product is suspended in 200 ml of DMF and added with 150 gr of the adduct pyridine-SO₃ dissolved in 200 ml of DMF. The solution is kept at 45° C. for 18 hours.

At the end of the reaction the solution is cooled to room temperature and added with 1,200 ml of acetone saturated with sodium chloride.

The pellet obtained is separated from the solvent by filtration, dissolved with 100 ml of deionised water and sodium chloride is added to 0.2M concentration. The solution is brought to pH 7.5-8 with 2N sodium hydroxide and 300 ml of acetone are added. The pellet is separated by filtration. The solid obtained is solubilised with 100 ml deionised water and purified from the residual salts by diafiltration as described in step i).

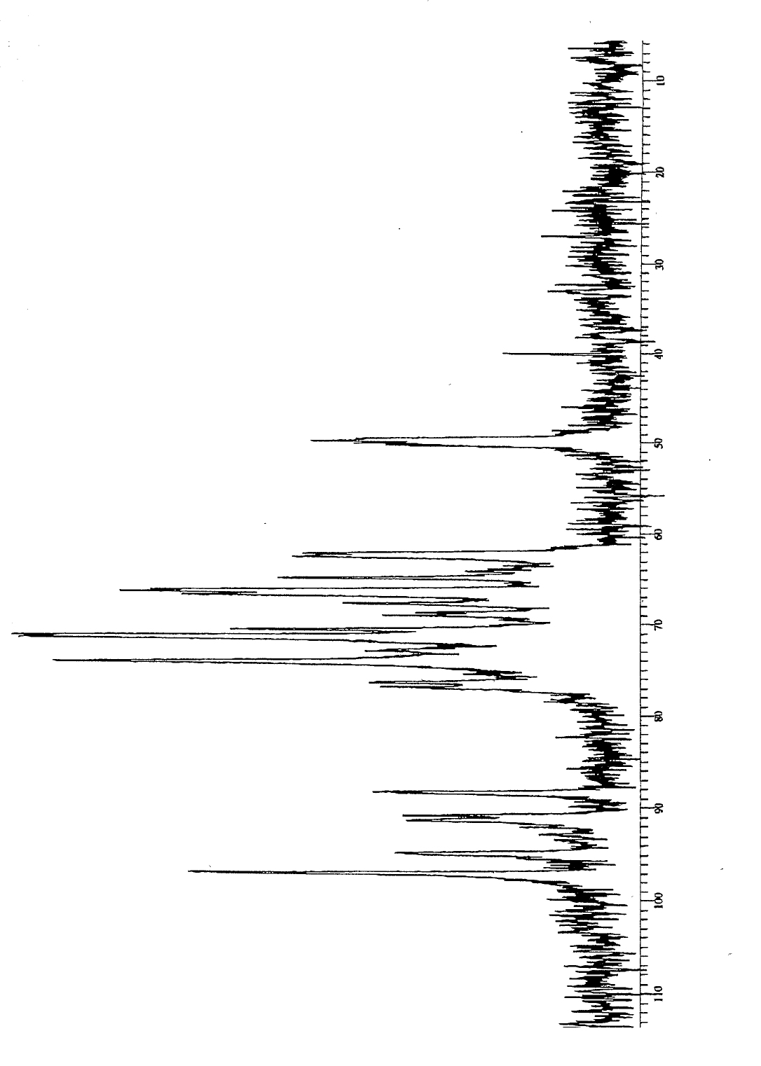

The ¹³C-NMR analysis on a dried small amount of the oversulfated product is shown in FIG. 13.

iv. The solution containing the product of step iii) is passed onto a IR 120 H⁺ cationic exchange resin (50 ml). After the passage of the solution the resin is washed with 3 volumes of deionised water. The pH of the flow through is more than 6. The acidic solution is brought to neutrality with pyridine. The solution is concentrated to 1/10 of the volume in a rotating evaporator at 40° C. under vacuum and freeze dried.

The product obtained as pyridine salt is added with 500 ml of a solution of DMSO/methanol (9/1 V/V). The solution is kept at 60° C. for 2.5 hours and then added with 50 ml deionised water and finally treated with 1,650 ml acetone saturated with sodium chloride.

The solid obtained is purified by diafiltration as described in step i) and a solution at 10% concentration is obtained.

The ¹³C-NMR analysis on a dried small amount in FIG. 13 shows a content of sulfate groups in position 6 of the amino sugar of 20%.

v. The solution containing the product of step iv) is passed onto a IR 120 H⁺ cationic exchange resin (50 ml). After the passage of the solution the resin is washed with 3 volumes of deionised water. The pH of the flow through is more than 6. The acidic solution is brought to neutrality with an aqueous solution of 15% tetrabutylammoniun hydroxide. The solution is concentrated to 1/10 of the volume in a rotating evaporator under vacuum and freeze dried. The product as tetrabutylammonium salt is suspended in 200 ml DMF. The suspension is cooled to 0° C. and treated with 40 gr. of the adduct pyridine-SO₃ dissolved in 100 ml DMF.

The sulfating agent is added one step.

The solution is kept at 0° C. for 1.5 hours and then is treated with 750 ml acetone saturated with sodium chloride.

The solid obtained is purified by diafiltration as described in step i).

vi. The solution of step v) is treated as described in step i) for N-sulfation.

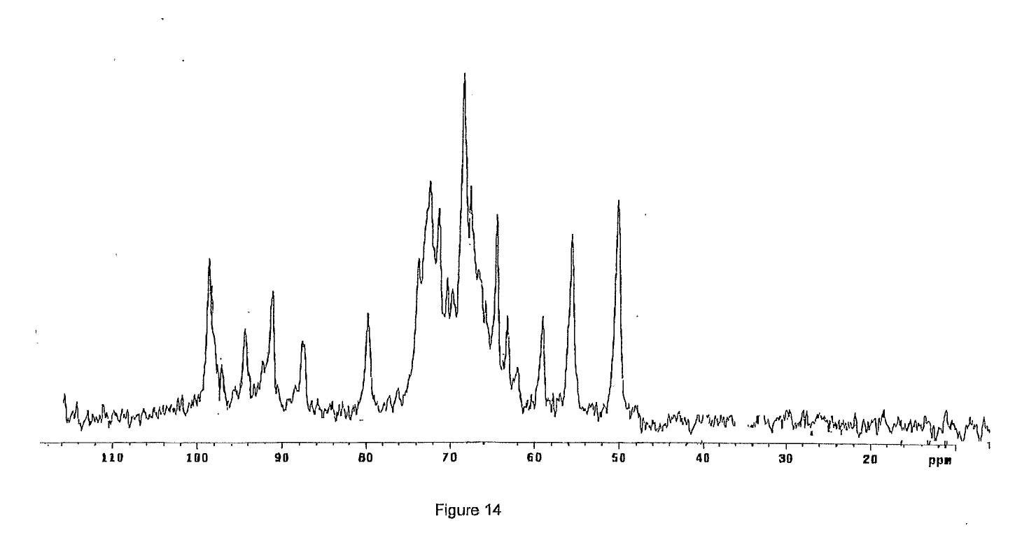

The ¹³C-NMR on a dried small amount of the product obtained is shown in FIG. 14.

The compound obtained shows a mean molecular weight of 15,700 (see reference b in table 1 and 2), sulfate/carboxyl ratio of 2.55, iduronic acid content of 54%, N-sulfate content of >90% . 6-O sulfate content of 85% , 3-O sulfate glucosamine content of 30%, 2-O sulfate content of 40% and 3-O sulfate uronic acid of 10%, an ATIII high affinity fraction of 55% and the following in vitro anticoagulant activities compared to heparin taken as 100.

| | |
|---|---|
| Anti-Xa | 157% |
| APTT | 78% |
| Anti-II | 373% |
| HCII | 161% |

### EXAMPLE 13

The example 12 is repeated and the compound is depolymerised as described in example 11.

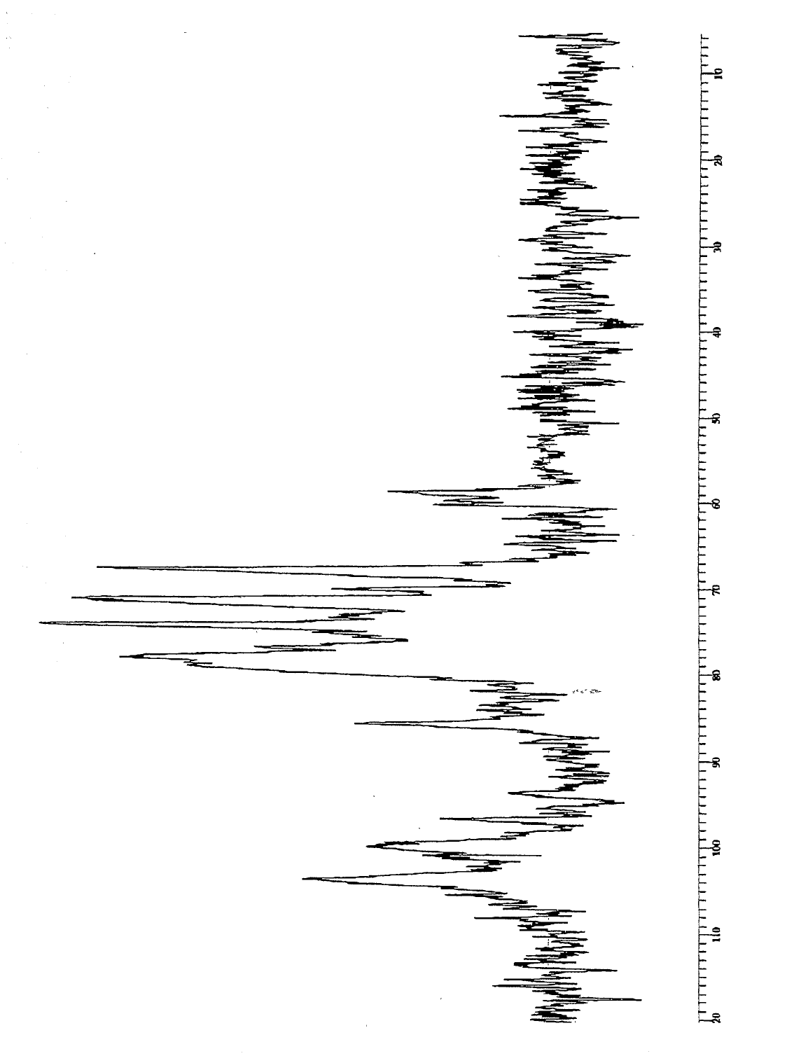

The compound obtained shows a ¹³C-NMR spectrum if FIG. 15, a mean molecular weight of 7,400, sulfate/carboxyl ratio of 2.55 , iduronic acid content of 54% , N-sulfate content of >90% , 6-O sulfate content of 85% , 3-O sulfate glucosamine content of 30%, 2-O sulfate content of 40% and 3-O sulfate uronic acid of 10%, an ATIII high affinity fraction of 34% and the following in vitro anticoagulant activities compared to heparin taken as 100.

| | |
|---|---|
| Anti-Xa | 99 |
| APTT | 52 |
| Anti-II | 203 |
| HCII | 108 |

### EXAMPLES 14-16

By operating as described in example 13 starting from the products of example 4, 6, 7, glycosaminoglycans are obtained having respectively the characteristics shown in table 3. Values represent a percentage against heparin (4^{th} Int. Std) taken as 100. It results from this table that the glycosaminoglycan of example 13 has a biochemical activity better than that of all the other low molecular weight glycosaminoglycans.

TABLE 3

| | Anti Xa % | APTT % | Anti IIa % | HCII % |
|---|---|---|---|---|
| Example 13 | 99 | 52 | 203 | 108 |
| Example 14 | 25 | 26 | 36 | 51 |
| Example 15 | 40 | 41 | 36 | 91 |
| Example 16 | 35 | 35 | 58 | 48 |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of all applications, patents and publications, cited above, and a corresponding Italian application filed March 2000, the assignee of record being INALCO, are hereby incorporated by reference.

From the foregoing description, one skilled in the art can easily ascertain the essential characteristics of this invention, and without departing from the spirit and scope thereof, can make various changes and modifications of the invention to adapt it to various usages and conditions.

1. N-deacetylate N-sulfate derivatives of K5 polysaccharide, epimerised at least till 40% of iduronic acid with respect to the total uronic acids, having molecular weight from 2,000 to 30,000 D, containing from 25 to 50% on weight of the chains with high affinity for ATIII and having an anticoagulant and antithrombotic activity expressed as ratio HCII/Anti-Xa comprised between 1.5 and 4.

2. Derivatives according to claim 1 characterised by the molecular weight comprised between 4,000 and 8,000 D.

3. Derivatives according to claim 1 characterised by the molecular weight comprised between 18,000 and 30,000 D.

4. Process for the preparation of derivatives of K5 polysaccharide as defined in claim 1, comprising in sequence the preparation of K5 polysaccharide from Escherichia Coli, N-deacetylation and N-sulfation, C5 epimerisation of D-glucuronic acid to L-iduronic acid, oversulfation, selective O-desulfation, selective 6-O sulfation and N-sulfation, characterised by the fact that said C5 epimerisation is performed using the enzyme glucuronosyl C5 epimerase in solution or in immobilised form in presence of specific divalent cations.

5. Process according to claim 4 characterised by the fact that said enzyme is chosen from the group comprising recombinant glucuronosyl C5 epimerase, glucuronosyl C5 epimerase from murine mastocytoma and glucuronosyl C5 epimerase extracted from bovine liver.

6. Process according to claim 4 characterised by the fact that said divalent cations are chosen from the group comprising Ba, Ca, Mg, Mn and are using alone or in combination.

7. Process according to claims 4 and 6 characterised by the fact that said C5 epimerisation with the enzyme in solution is performed dissolving an amount of enzyme C5 epimerase comprised between 1.2×10⁷ and 1.2×10¹¹ cpm in 2-2,000 ml of 25 mM Hepes buffer at a pH between 5.5 and 7.4 containing from 0.001 to 10 gr. of N-deacetylate N-sulfate K5 and one or a combination of said cations at a concentration comprised between 10 and 60 mM.

8. Process according to claim characterised by the fact that said C5 epimerisation with the enzyme in solution is performed at a temperature between 30 and 40° C. for a time comprised between 1 and 24 hours.

9. Process according to claims from 4 to 6 characterised by the fact that said C5 epimerisation with the enzyme in its immobilised form is performed recirculating 20-1,000 ml of a solution of 25 mM Hepes at pH from 6 to 7.4 containing 0.001-10 gr of N-deacetylate N-sulfate K5 and one of said cations at a concentration between 10 and 60 mM through a column containing from 1.2×10⁷ to 3×10¹¹ cpm of the immobilised enzyme on an inert support.

10. Process according to claim 9 characterised from the fact that said C5 epimerisation is performed at a temperature between 30 and 40° C. recirculating said solution with a flow rate of 30-160 ml/hour for a time between 1 and 24 hours.

11. A glycosaminoglycan constituted by a mixture of chains of the general structure: 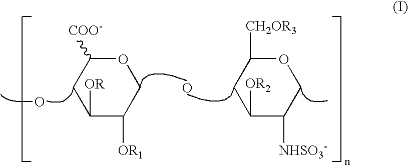
wherein at least 40% of the uronic moieties being those of iduronic acid, R, R₁, R₂ and R₃ represent a hydrogen atom or a SO₃⁻ group and n is an integer of from 3 to 100, 10% of R, R₁, R₂ and R₃ being hydrogen and the remaining being SO₃⁻ groups distributed as follows: from about 90 to about 100% in R₃, from about 25 to about 30% in R₂, from about 30 to about 50% in R₁ and about 10% R, the sulfation degree being front about 2.3 to about 2.6 and the corresponding cation being a pharmaceutically acceptable one.

12. The glycosaminoglycan of claim 11 in which the corresponding cation is selected from the group consisting of alkaline metal, alkaline-earth metal, ammonium, (C₁C₄)trialkylammonium, aluminium and zinc ions.

13. The glycosaminoglycan of claim 12 in which the corresponding cation is the sodium or calcium ion.

14. A glycosaminoglycan according to claim 11 in which said mixture of chains contains at least 80% of chains of formula I wherein n is from 3 to 15.

15. The glycosaminoglycan of claim 14 in which said mixture of chains has a molecular weight distribution ranging from about 2,000 to about 10,000, with a mean molecular weight of from about 4,000 to about 8,000.

16. The glycosaminoglycan of claim 15 in which said mixture of chains having said molecular weight distribution is a mixture of chains having the structure 1 in which at least 50% of the uronic moieties being those of iduronic acid and
R is for about 80% hydrogen and for about 20% a SO₃⁻ group;
R₁ is for about 60% hydrogen and for about 40% a SO₃⁻ group;
R₂ is for about 70% hydrogen and for about 30% a SO₃⁻ group;
R₃ is for about 10% hydrogen and for about 90% a SO₃⁻ group;
the sulfation degree being about 2.5.

17. The glycosaminoglycan of claim 11 in which said mixture of chains contains at least 80% of chains of formula I wherein n is from 20 to 100.

18. A glycosaminoglycan according to claim 11 in which said mixture of chains has a molecular weight distribution ranging from about 9,000 to about 60,000, with a mean molecular weight of from about 12,000 to about 30,000.

19. The glycosaminoglycan of claim 18 in which said mixture of chains having said molecular weight distribution is a mixture of chains having the structure 1 in which at least 50% of the uronic moieties being those of iduronic acid and
R is for about 80% hydrogen and for about 20% a SO₃⁻ group;
R₁ is for about 60% hydrogen and for about 40% a SO₃⁻ group;
R₂ is for about 70% hydrogen and for about 30% a SO₃⁻ group;
R₃ is for about 10% hydrogen and for about 90% a SO₃⁻ group;
the sulfation degree being about 2.5.

20. The glycosaminoglycan of claim 18 in which the corresponding cation is selected from the group consisting of alkaline metal, alkaline-earth metal, ammonium, (C₁C₄)trialkylammonium, aluminium and zinc ions.

21. The glycosaminoglycan of claim 20 in which the corresponding cation is the sodium or calcium ion.

22. The glycosaminoglycan of claim 19 in which the corresponding cation is selected from the group consisting of alkaline metal, alkaline-earth metal, ammonium, (C₁C₄)trialkylammonium, aluminium and zinc ions.

23. The glycosaminoglycan of claim 22 in which the corresponding cation is the sodium or calcium ion.

24. A pharmaceutical composition comprising, as an active ingredient, a glycosaminoglycan according to claim 11 and a pharmaceutically acceptable carrier.

25. The composition of claim 24 comprising, as an active ingredient, a glycosaminoglycan according to claim 20 and a pharmaceutically acceptable carrier.

26. The composition of claim 24 comprising, as an active ingredient, a glycosaminoglycan according to claim 22 and a pharmaceutically acceptable carrier.

27. A process for the preparation of K5 glycosaminoglycans comprising the steps of (i) N-deacetylation/N-sulfation of the polysaccharide K5, (ii) partial C-5 epimerisation of the carboxyl group of the glucuronic acid moiety to the corresponding iduronic acid moiety, (iii) oversulfation, (iv) selective O-desulfation, (v) optional selective 6-O-sulfation, and (vi) N-sulfation, in which step (iv) comprises treating the oversulfated product obtained at the end of step (iii) with a mixture methanol/dimethyl sulfoxide for a period of time of from 135 to 165 minutes.

28. The process of claim 27 in which said period of time is of about 150 minutes.

29. The process of claim 27 in which said treatment is made for a period of time of about 150 minutes at a temperature of about 60° C.

30. A method for treating thrombosis in a mammal which comprises administering to said mammal an effective amount of glycosaminoglycan as claimed in claim 11.

31. The method of claim 30 in which said effective amount is administered in pharmaceutical composition containing from 5 to 100 mg of said glycosaminoglycan.
